(19) United States
(12) Patent Application Publication
(10) Pub. No.: US 2003/0228019 A1
(43) Pub. Date: Dec. 11, 2003

(54) Method and system for reducing noise

(75) Inventors: Uzi Eichler, Haifa (IL); Lior Barak, Haifa (IL); Avner Paz, Reut (IL)

Correspondence Address: TESTA, HURWITZ & THIBEAULT, LLP, HIGH STREET TOWER, 125 HIGH STREET, BOSTON, MA, 02110, US

(73) Assignee: Elbit Systems Ltd., Haifa (IL)

(21) Appl. No.: 10/167,066

(22) Filed: Jun. 11, 2002

(51) Int. Cl. A61F 11/06; G10K 11/16; H03B 29/00

(52) U.S. Cl. 381/71.8; 381/71.1; 381/57

(57) ABSTRACT

System for producing a substantially noise-free signal of an acoustic sound, and for producing a sound, the sound including a desired sound and an anti-phase noise sound, the anti-phase noise sound being in anti-phase relative to a noise, the system including an acoustoelectric transducer, a reference-acoustoelectric transducer and an audio controller coupled with the reference-acoustoelectric transducer and the acoustoelectric transducer, wherein the acoustoelectric transducer produces a noise bearing sound signal by detecting the acoustic sound and the noise, wherein the reference-acoustoelectric transducer produces the reference noise signal by detecting the noise in a noisy environment and wherein the audio controller produces the substantially noise-free signal, according to the reference noise signal and the noise bearing sound signal.

### FIELD OF THE DISCLOSED TECHNIQUE

The disclosed technique relates to audio systems in general, and to methods and systems for reducing background noise, in particular.

### BACKGROUND OF THE DISCLOSED TECHNIQUE

Ambient noise from various sources creates a noisy environment that often amounts to a disturbance to a person or an acoustic receiver. The noise may considerably interfere with sounds that are required to be captured by an acoustic sensor, or distract a person who is required to concentrate on specific tasks. Tasks that require listening to sounds by a human ear or their capturing via a microphone, are particularly vulnerable to disruption by noise. In this context, the noise is an objectionable acoustic pressure impinging upon the eardrums of a person or upon the receiving means of an acoustic sensor.

Devices and methods in the prior art were designed to provide active attenuation of noise. U.S. Pat. No. 4,985,925 issued to Langberg et al., provides an active noise reduction based on a negative feedback electro-acoustical system. The electro-acoustical system consists of an electronic earplug seated in the concha fossa. The system combines active and passive noise reduction in the quiet zone of the ear, a bilateral transducer circuit, a shunt feedback control filter network, and a combined input noise-filter/feedback system. The bilateral transducer circuit drives a speaker as an acoustical velocity source. The shunt feedback control filter network improves stability and increases noise reduction.

U.S. Pat. No. 5,600,729 issued to Darlington et al., teaches the application of Active Noise Reduction (ANR) in an ear defender. The ear defender includes detector means (e.g., a microphone) for detecting the sound level in the proximity of the ear of the person. The ear defender further includes output means (e.g., a speaker) for generation of noise reduction signal within the ear shell. The ear defender also includes a digital feedback controller for generating a feedback signal derived from the output of the detector means and applying it to the output means. The ear defender also features estimation means for providing estimation of the ear shell transfer function and subtracting from the input to the feedback controller, a signal representing the estimated electroacoustic transfer function of the system. A second, analog or digital feedback controller provides an active noise control on the basis of an average configuration for the system.

U.S. Pat. No. 6,078,672 issued to Saunders et al., provides a personal noise attenuation system for attenuating both tonal and broadband sound in a noisy environment immediately adjacent to a user. The system includes a spatially adjustable acousto-electric sensor adapted to sense ambient noise, including certain preselected sounds. The system also has attenuation means including both feedback and feed forward components so as to provide a heteronomous attenuation and more complete active noise attenuation. The adjustable acoustoelectric sensor is spatially moved so as to adapt to the changing physical characteristics of spatial zones in different noise fields adjacent to the user.

U.S. Pat. No. 6,278,786 issued to McIntosh, provides an active noise cancellation aircraft headset system. A speaker is mounted within each earcup of a headset for receiving and acoustically transducing a composite noise cancellation signal. A microphone is also mounted within each earcup for transducing acoustic pressure within the earcup to a corresponding analog error signal. An analog filter receives the analog error signal and inverts it to generate an analog broadband noise cancellation signal. The analog error signal is also provided to an analog to digital converter, which receives the analog microphone error signal and converts it to a digital error signal.

A digital signal processor (DSP) takes the digital error signal and using an adaptive digital feedback filter, generates a digital tonal noise cancellation signal. A digital to analog converter then converts the digital tonal noise cancellation signal to an analog tonal noise cancellation signal so that it can be summed with the analog broadband noise cancellation signal to form a composite cancellation signal. The composite cancellation signal is provided to the speakers in the earcups to cancel noise within the earcups. The broadband analog cancellation is effective to reduce overall noise within the earcup. The DSP provides active control of the analog cancellation loop gain to maximize the effectiveness of the broadband analog cancellation. The DSP also uses the adaptive feedback filter/algorithm to substantially reduce at least the loudest tonal noises penetrating the earcup. The tonal noses include engine and propeller noises, as well as harmonic vibrations of components of the fuselage of the aircraft.

### SUMMARY OF THE DISCLOSED TECHNIQUE

It is an object of the disclosed technique to provide a novel method and system for producing a noise-free sound signal of the voice of a person talking in a noisy environment, which overcomes the disadvantages of the prior art.

In accordance with the disclosed technique, there is thus provided a system for producing a substantially noise-free signal of an acoustic sound (e.g., the voice of a pilot transmitting to an air traffic controller). The system furthermore produces a sound which includes a desired sound (e.g., the voice of an air traffic controller transmitted to the pilot) and an anti-phase noise sound, the anti-phase noise sound being in anti-phase relative to a noise. The system includes an acoustoelectric transducer, a reference-acoustoelectric transducer and an audio controller coupled with the reference-acoustoelectric transducer and the acoustoelectric transducer.

The acoustoelectric transducer produces a noise bearing sound signal by detecting the acoustic sound and the noise, and the reference-acoustoelectric transducer produces the reference noise signal by detecting the noise in a noisy environment. The audio controller produces the substantially noise-free signal, according to the reference noise signal and the noise bearing sound signal.

The system further includes an electroacoustic transducer for producing the sound and an active noise reduction controller coupled with the electroacoustic transducer and the reference-acoustoelectric transducer. The active noise reduction controller produces a sound signal according to the reference noise signal and according to a desired sound signal respective of the desired sound. The electroacoustic transducer produces the sound according to the sound signal.

In accordance with another aspect of the disclosed technique, there is thus provided a system for producing a sound. The sound includes a desired sound (e.g., the voice of an air traffic controller transmitted to a pilot) and an anti-phase noise sound, the anti-phase noise sound being in anti-phase relative to a noise. The system includes an electroacoustic transducer, a reference-acoustoelectric transducer and an active noise reduction controller coupled with electroacoustic transducer and the reference-acoustoelectric transducer.

The electroacoustic transducer produces the sound and the reference-acoustoelectric transducer produces a reference noise signal by detecting the noise in a noisy environment. The active noise reduction controller produces a sound signal according to the reference noise signal and according to a desired sound signal respective of the desired sound, and the electroacoustic transducer produces the sound according to the sound signal.

In accordance with a further aspect of the disclosed technique, there is thus provided a system for producing an anti-phase noise sound. The system includes an electroacoustic transducer, a reference-acoustoelectric transducer for producing a reference noise signal by detecting noise in a noisy environment and a digital active noise reduction controller coupled with the electroacoustic transducer and the reference-acoustoelectric transducer.

The digital active noise reduction controller produces an anti-phase noise signal according to the reference noise signal, wherein the anti-phase noise signal is in anti-phase relative to the reference noise signal. The electroacoustic transducer produces the anti-phase noise sound according to the anti-phase noise signal.

In accordance with another aspect of the disclosed technique, there is thus provided a system for producing sound, the sound including a desired sound (e.g., the voice of an air traffic controller transmitted to a pilot) and an anti-phase noise sound, the anti-phase noise sound being in anti-phase relative to a noise. The system includes an electroacoustic transducer, a reference-acoustoelectric transducer, an error-acoustoelectric transducer, a feedforward element and a feedback element. The system further includes a first summing element, a second summing element, a third summing element, a first estimated plant response element and a second estimated plant response element.

The reference-acoustoelectric transducer produces a reference noise signal by detecting the noise in a noisy environment. The feedforward element is coupled with the reference-acoustoelectric transducer. The feedback element is coupled with the feedforward element. The first summing element is coupled with the feedforward element, the feedback element and with the electroacoustic transducer. The second summing element is coupled with the feedback element, the feedforward element and with the error-acoustoelectric transducer. The third summing element is coupled with the feedback element and with the second summing element. The first estimated plant response element is coupled with the second summing element and the second estimated plant response element is coupled with the third summing element and with the electroacoustic transducer.

The first summing element produces a summation signal, by adding a feedback signal received from the feedback element, a feedforward signal received from the feedforward element, and a sound signal respective of the desired sound. The electroacoustic transducer produces the sound according to the summation signal. The first estimated plant response element produces a first estimated desired sound signal, respective of the desired sound as produced by the electroacoustic transducer.

The error-acoustoelectric transducer produces an error signal by detecting the sound. The second summing element produces a first difference signal, by subtracting the first estimated desired sound signal from the error signal. The second estimated plant response element produces an estimated difference signal, according to the summation signal. The third summing element produces a second difference signal, by subtracting the estimated difference signal from the first difference signal. The feedback element produces the feedback signal according to the first difference signal and the second difference signal and the feedforward element produces the feedforward signal, according to the reference noise signal and the first difference signal.

In accordance with a further aspect of the disclosed technique, there is thus provided a method for producing a noise-free sound signal. The method includes the procedures of producing a noise bearing sound signal by detecting acoustic sound and noise, producing a reference noise signal by detecting noise, determining a correction signal according to the reference noise signal and producing the noise-free sound signal, according to the noise bearing sound signal and the correction signal.

In accordance with another aspect of the disclosed technique, there is thus provided a method for producing a noise-canceling sound. The method includes the procedures of producing a reference noise signal by detecting noise, determining a noise-canceling signal according to the reference noise signal and producing the noise-canceling sound according to the determined noise-canceling signal.

In accordance with a further aspect of the disclosed technique, there is thus provided a method for producing an audio-and-noise-canceling sound. The method includes the procedures of producing a reference noise signal by detecting noise, receiving an audio signal, determining an audio-and-noise-canceling signal according to the reference noise signal and the audio signal, and producing the audio-and-noise-canceling sound according to the determined audio-and-noise-canceling signal.

### BRIEF DESCRIPTION OF THE DRAWINGS

The disclosed technique will be understood and appreciated more fully from the following detailed description taken in conjunction with the drawings in which:

FIG. 1A is a schematic illustration of a system for producing a noise-free sound signal, constructed and operative in accordance with an embodiment of the disclosed technique;

FIG. 1B is a schematic illustration of a detail of the audio controller of the system of FIG. 1A;

FIG. 1C is a schematic illustration of the system of FIG. 1A incorporated with a head-mounted device;

FIG. 2A is a schematic illustration of a noise-canceling system, constructed and operative in accordance with another embodiment of the disclosed technique;

FIG. 2B is a schematic illustration of a detail of the analog ANR controller of the ANR controller of the system of FIG. 2A;

FIG. 2C is a schematic illustration of the system of FIG. 2A, incorporated with a head-mounted device;

FIG. 3A is a schematic illustration of a noise reduction system, constructed and operative in accordance with a further embodiment of the disclosed technique;

FIG. 3B is a schematic illustration of the system of FIG. 3A, incorporated with a head-mounted device;

FIG. 4A is a schematic illustration of a digital noise reduction system, constructed and operative in accordance with another embodiment of the disclosed technique;

FIG. 4B is a schematic illustration of the feedforward portion of the system of FIG. 4A;

FIG. 4C is a schematic illustration of the feedback portion of the system of FIG. 4A;

FIG. 5A is a schematic illustration of a method for operating the system of FIG. 1A, operative in accordance with a further embodiment of the disclosed technique;

FIG. 5B is a schematic illustration of a method for operating a noise-canceling system, operative in accordance with another embodiment of the disclosed technique; and

FIG. 6 is a schematic illustration of a method for operating the system of FIG. 3A, operative in accordance with a further embodiment of the disclosed technique.

### DETAILED DESCRIPTION OF THE EMBODIMENTS

The disclosed technique processes a background noise signal together with a signal containing the background noise and a desired sound, and produces a signal of the desired sound, substantially free of the background noise. The disclosed technique produces a noise-free signal from the voice of a person speaking in a noisy environment. The disclosed technique allows a person located in a noisy environment, to hear the desired sound, substantially free from noise.

The term “acoustoelectric transducer” herein below, refers to a device which converts acoustical signals to electrical signals (e.g., a microphone). The term “electroacoustic transducer” herein below, refers to a device which converts electrical signals to acoustical signals (e.g., a loudspeaker). An acoustoelectric transducer can operate based on principles of electrodynamics, electrostatics, piezoelectricity, magnetostriction, fiber-optics, stimulation of carbon particles, and the like. An electroacoustic transducer can operate based on principles of electrodynamics, magnetism, piezoelectricity, magnetostriction, hydraulic, and the like. The term “electric” herein includes all electromagnetic signals, such as electric, optic, radio, and the like, that can be transmitted by wire or other communication channels, or wirelessly.

The term “quiet zone” herein below, refers to a region in the vicinity of the ear-drum, the ear, or within the outer canal thereof, at which a sound at approximately 180 degrees out-of-phase relative to the ambient noise (anti-phase, or out-of-phase by π radians), cancels the ambient noise and as a result, the person does not hear the ambient noise. The locations “close to the ear” herein below, are approximate and refer to the quiet zone. The term “tonal noise” herein below, refers to a noise which is confined to substantially limited frequency range or ranges, such as the noise generated by the rotors of a helicopter.

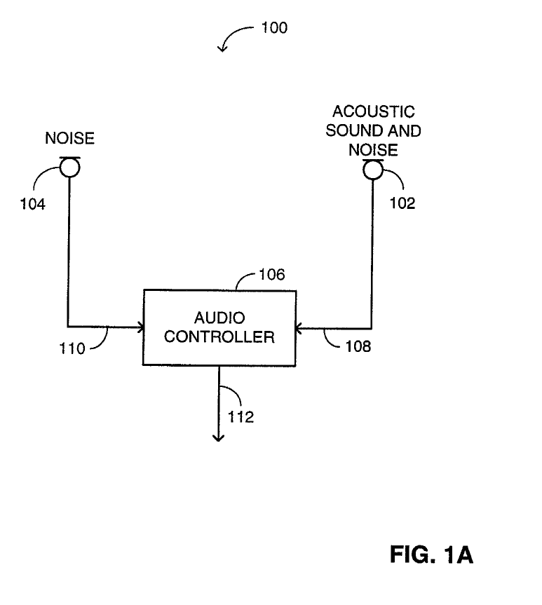

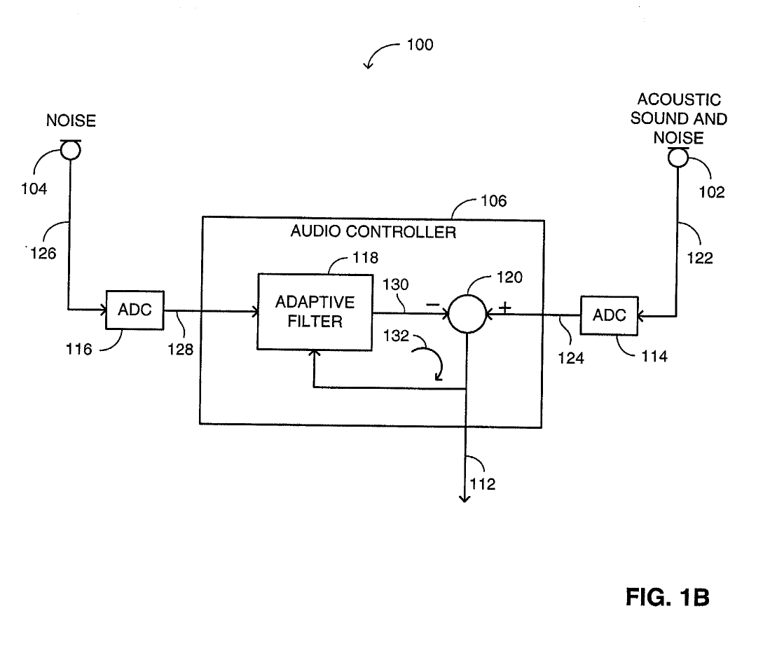

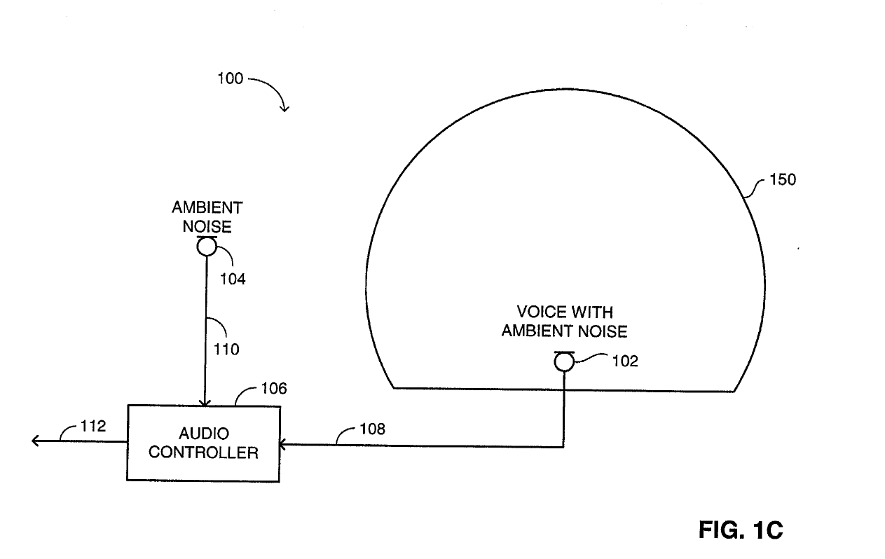

Reference is now made to FIGS. 1A, 1B and 1C. FIG. 1A is a schematic illustration of a system for producing a noise-free sound signal, generally referenced 100, constructed and operative in accordance with an embodiment of the disclosed technique. FIG. 1B is a schematic illustration of a detail of the audio controller of the system of FIG. 1A. FIG. 1C is a schematic illustration of the system of FIG. 1A incorporated with a head-mounted device, generally referenced 150.

With reference to FIG. 1A, system 100 includes acoustoelectric transducers 102 and 104 and an audio controller 106. Audio controller 106 is coupled with acoustoelectric transducers 102 and 104.

Audio controller 106 is a digital processor, which simultaneously samples two input signals at the same sampling rate and determines a transfer function for these two input signals, according to an adaptive filtering method. Audio controller 106 applies the transfer function on one of the input signals and subtracts the result from the other input signal. Audio controller 106, then produces an output signal respective of the result of the subtraction.

Acoustoelectric transducer 102 detects acoustic sound. This acoustic sound can be a human voice, machine generated voice, and the like. If the acoustic sound is the voice of a person (not shown), then acoustoelectric transducer 102 is located close to the mouth (not shown) of the person. Acoustoelectric transducer 102 detects the desired sound (i.e., the voice) as well as the noise (i.e., an undesired sound) which is present in the environment surrounding the person. The noise is generated for example, by other persons and devices, such as engines, turbines, motors, and mechanical devices, hydraulic or pneumatic devices (e.g., tubing, actuators), electromechanical devices (e.g., electric motor), loud-speakers which surround the speaker, firing of ammunition, by environmental sources, such as wind, rain, ocean waves, thunderstorm, by animals, and the like.

Acoustoelectric transducer 104 and acoustoelectric transducer 102 detect different sounds, due to either a sound absorbing material (not shown), located between acoustoelectric transducers 102 and 104, or the mere distance between acoustoelectric transducers 102 and 104. Thus, acoustoelectric transducer 104 detects the noise and substantially none of the desired sound, while acoustoelectric transducer 102 detects the desired sound and noise.

Audio controller 106 receives signals 108 and 110 from acoustoelectric transducers 102 and 104, respectively. Each of signals 108 and 110 is in analog format. An analog to digital converter (not shown) and herein below referred to as ADC, which converts an analog signal to a digital signal, is coupled with acoustoelectric transducer 102 and audio controller 106. Another ADC (not shown) is coupled with acoustoelectric transducer 104 and audio controller 106. Thus, audio controller 106 receives signals 108 and 110 which are in digital format.

Signal 108 includes information respective of a desired sound and noise. Signal 110 includes information respective of noise. Audio controller 106 determines a new reduced-intensity sound pressure level (SPL) for signal 110, by employing an SPL converter (not shown). The SPL converter can be in form of a hardwired look-up table, a software look-up table, a hardwired transfer function, a software transfer function, an adaptive filter, and the like. Audio controller 106 subtracts the new determined SPL from the SPL of signal 108, which corresponds to signal 110. The noise detected by acoustoelectric transducer 102 is different from the noise detected by acoustoelectric transducer 104, namely—it is usually at a reduced intensity and with a retarded phase (due to an acoustic insulation or acoustic insulating distance between acoustoelectric transducers 102 and 104). Thus, the new determined SPL corresponds to a reduced and retarded function of the SPL of signal 110. Audio controller 106 produces a signal 112 respective of the result of the above subtraction operation. Thus, signal 112 includes information respective of the desired sound, substantially excluding the noise.

The form and the parameters of the SPL converter are determined in accordance with certain physical parameters, such as the hearing characteristics of a person, the voice characteristics of a person, the sound absorbing characteristics of a headset worn by a person, the dimensions of the headset, the relative distances between acoustoelectric transducer 102 and acoustoelectric transducer 104, the acoustic properties of the environment which surround acoustoelectric transducer 102 and acoustoelectric transducer 104, the acoustic properties of the sound absorbing material located between acoustoelectric transducer 102 and acoustoelectric transducer 104, and the like.

With reference to FIG. 1B, system 100 includes acoustoelectric transducers 102 and 104, audio controller 106 and analog to digital converters 114 and 116. Audio controller 106 includes an adaptive filter 118 and a summing element 120. ADC 114 is coupled with acoustoelectric transducer 102 and summing element 120. ADC 116 is coupled with acoustoelectric transducer 104 and adaptive filter 118. Alternatively, ADC 114 is integrated with either acoustoelectric transducer 102 or audio controller 106. Similarly, ADC 116 can be integrated with acoustoelectric transducer 104 or audio controller 106.

Acoustoelectric transducer 102 produces an analog signal 122 and sends analog signal 122 to ADC 114. ADC 114 converts analog signal 122 to a digital signal 124, sends digital signal 124 to summing element 120 and adaptive filter 118 produces a signal 130 according to signal 128. Signal 130 is respective of the ambient noise detected by acoustoelectric transducer 104 at a reduced SPL (i.e., the SPL of the ambient noise close to acoustoelectric transducer 102). Summing element 120 produces signal 112 by subtracting signal 130 from signal 124. Signal 112 is further provided to an interface (not shown) for further processing or transmission. Acoustoelectric transducer 104 produces an analog signal 126 and sends analog signal 126 to ADC 116. ADC 116 converts analog signal 126 to a digital signal 128 and sends digital signal 128 to adaptive filter 118. Signal 112 from summing element 120 is fed back to adaptive filter 118, in a feedback loop 132. If signal 112 includes any residual noise, then adaptive filter 118 detects this residual noise and adjusts signal 130 accordingly. Summing element 120 then subtracts this residual noise from signal 124.

With reference to FIG. 1C, acoustoelectric transducer 102 is incorporated with head-mounted device 150. Audio controller 106 is coupled with acoustoelectric transducers 102 and 104. Head-mounted device 150 is in form of a helmet, a headset, and the like. Acoustoelectric transducer 102 is located at the mouth (not shown) of the user (not shown). Acoustoelectric transducer 104 is located external to head-mounted device 150 or externally mounted thereon, but acoustically insulated or remote from the mouth of the user.

Head-mounted device 150 can include a visual device (not shown), such as a head-up display, visor, liquid crystal display (LCD), field emission display (FED), mirror, and the like. Additionally, head-mounted device 150 can include one or more electroacoustic transducers.

If head-mounted device 150 is in form of a helmet, it can include sound absorbing material, such as mineral wool, fiberglass, and the like. In this case, acoustoelectric transducer 102 detects the voice of the user, while also detecting the background noise—but at a reduced SPL.

In case head-mounted device 150 is in form of a headset, due to the physical distance of acoustoelectric transducer 104 from the mouth of the user, acoustoelectric transducer 104 detects the ambient noise and substantially none of the voice of the user. However, acoustoelectric transducer 102 detects the voice of the user and the ambient noise. It is noted that even ambient air can effectively acoustically insulate, such as insulating acoustoelectric transducer 104 from the mouth of the user.

In case head-mounted device 150 is a helmet worn by a pilot (not shown), the ambient noise can be the noise generated by the engine (i.e., power-plant) of the aircraft, by the engines of other aircraft flying closeby, the voices of the aircraft crew, the sound of thunder, the sound of ice particles striking the windshield, the sound of firing ammunition, and the like. Acoustoelectric transducer 102 is attached to the inner portion of head-mounted device 150, close to the mouth of the pilot and acoustoelectric transducer 104 is attached to the outer portion of head-mounted device 150.

Head-mounted device 150 includes sound absorbing material, and acoustoelectric transducer 104 is farther away from the mouth of the pilot than acoustoelectric transducer 102. Hence, acoustoelectric transducer 104 detects mostly the ambient noise and substantially none of the voice of the pilot. However, since the sound absorbing material of head-mounted device 150 absorbs only a portion of the sound, acoustoelectric transducer 102 detects the voice of the pilot, in addition to the ambient noise at a reduced SPL. Thus, signal 108 includes information respective of the voice of the pilot and an attenuated level of the ambient noise, while signal 110 includes information respective of the ambient noise at an SPL higher than that detected by acoustoelectric transducer 102. The attenuation level of the ambient noise may depend on frequency.

The parameters of the SPL converter can be determined empirically, by measuring the SPL values of signals 108 and 110 in a selected frequency range, in response to sound corresponding to the SPL values and in the frequency range of the expected ambient noise. It is noted that these measurements are performed without the voice of the pilot in the same location within the aircraft, in which system 100 is employed. These measurements can be performed before flight as “pre-calibrations” or during speech pauses at flight time. In addition, audio controller 106 calibrates system 100, at the beginning of every flight. Alternatively, the parameters of the SPL converter can be determined analytically, by computing the estimated attenuation of SPL values of the ambient noise in a selected frequency range.

It is further noted that the attenuated SPL value of the ambient noise detected by acoustoelectric transducer 102, depends also on the physical distance between acoustoelectric transducers 102 and 104. It is noted that due to the physical distance between acoustoelectric transducers 102 and 104 and a given value of the speed of sound, signals 108 and 110 can include information respective of the ambient noise waveform, which are out of phase. In order to subtract the correct portion of the ambient noise waveform from signal 108, audio controller 106 takes this phase-shift into account, by referring to a respective look-up table, transfer function, and the like.

According to another aspect of the disclosed technique, a noise reduction system employs an active noise reduction (ANR) controller, to produce a noise-free sound close to the ear of a user. The ANR controller produces an anti-phase signal of the ambient noise, which is derived from the detection of ambient noise by an external acoustoelectric transducer.

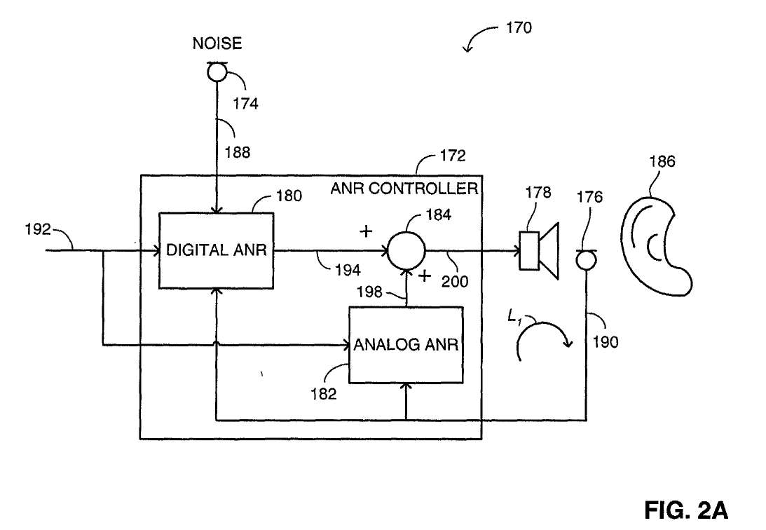

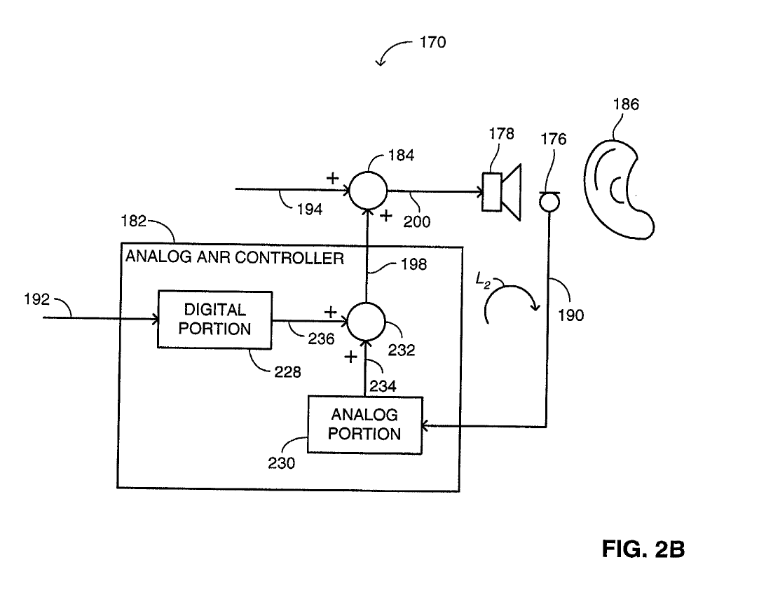

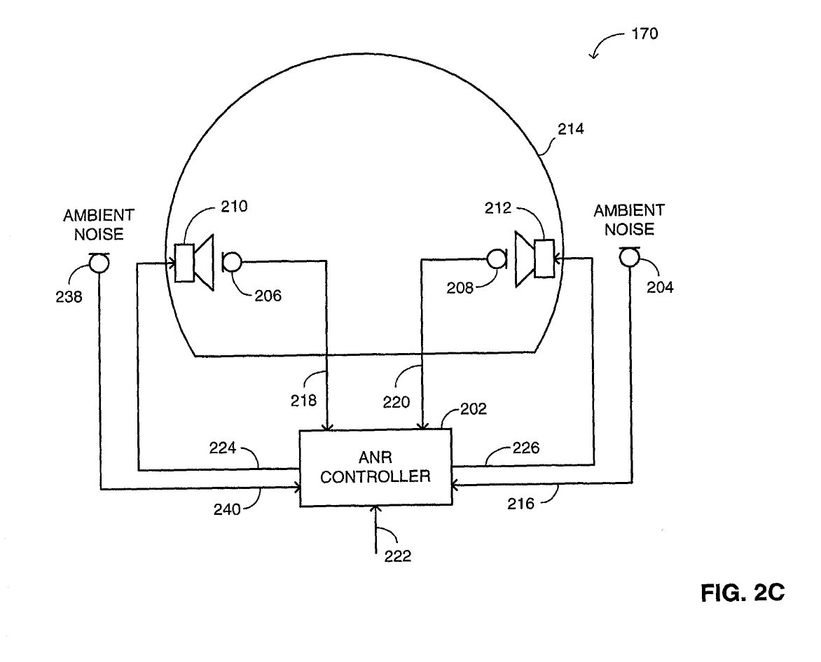

Reference is now made to FIGS. 2A, 2B and 2C. FIG. 2A is a schematic illustration of a noise-canceling system, generally referenced 170, constructed and operative in accordance with another embodiment of the disclosed technique. FIG. 2B is a schematic illustration of a detail of the analog ANR controller of the ANR controller of the system of FIG. 2A. FIG. 2C is a schematic illustration of the system of FIG. 2A, incorporated with a head-mounted device, generally referenced 214.

With reference to FIG. 2A, system 170 includes an ANR controller 172, a reference acoustoelectric transducer 174, an error acoustoelectric transducer 176 and an electroacoustic transducer 178. ANR controller 172 includes a digital ANR controller 180, an analog ANR controller 182 and a primary summing element 184. Digital ANR controller 180 is a device which produces an anti-phase signal for an input signal, at a reduced SPL. Analog ANR controller 182 is a device which produces an anti-phase signal for an input signal, at the same SPL.

Digital ANR controller 180 is coupled with reference acoustoelectric transducer 174, error acoustoelectric transducer 176 and with primary summing element 184. Analog ANR controller 182 is coupled with error acoustoelectric transducer 176 and with primary summing element 184. Primary summing element 184 is coupled with electroacoustic transducer 178.

Electroacoustic transducer 178 and error acoustoelectric transducer 176 are located close to an ear 186 of a user (not shown). Reference acoustoelectric transducer 174 is located substantially remote from ear 186. Alternatively, a sound absorbing material (not shown) is located between electroacoustic transducer 178 and error acoustoelectric transducer 176 on one side and reference acoustoelectric transducer 174 on the other. In both cases, reference acoustoelectric transducer 174 detects the ambient noise and substantially none of the sound produced by electroacoustic transducer 178. Likewise, error acoustoelectric transducer 176 detects the sound emitted by electroacoustic transducer 178 and the ambient noise at a location close to ear 186.

Following is a description of a loop L₁ formed by digital ANR controller 180, primary summing element 184, electroacoustic transducer 178 and error acoustoelectric transducer 176. Digital ANR controller 180 continuously samples a signal 188 from reference acoustoelectric transducer 174, respective of the ambient noise, and a signal 192 respective of a desired sound, from a sound source (not shown). The desired sound from the sound source can be a human voice, machine generated sound, mechanical voice, a sound signal, an acoustic sound (e.g., loud speaker), and the like.

Digital ANR controller 180 determines a reduced SPL for signal 188 by employing an SPL converter as described herein above in connection with audio controller 106 (FIG. 1C). The reduced SPL for signal 188 corresponds to the SPL of the ambient noise, at a location close to ear 186. Digital ANR controller 180 produces an anti-phase signal (not shown) for signal 188 at the reduced SPL, and adds this anti-phase signal at the reduced SPL, to signal 192, thereby producing a signal 194. Electroacoustic transducer 178 produces a sound according to signal 194.

It is noted that error electroacoustic transducer 176 is located sufficiently close to ear 186, such that the anti-phase sound of the ambient noise at the quiet zone of ear 186, which is emitted by electroacoustic transducer 178, substantially cancels out the ambient noise at the quiet zone of ear 186. Error acoustoelectric transducer 176 is located sufficiently close to electroacoustic transducer 178, to detect the sound emitted by electroacoustic transducer 178.

Digital ANR controller 180 receives a signal 190 from error acoustoelectric transducer 176, respective of the sound emitted by electroacoustic transducer 178 (which includes the desired sound and the anti-phase of the ambient noise at a location close to ear 186) and the ambient noise at a location close to ear 186. Digital ANR controller 180 modifies a portion of signal 194 respective of the anti-phase of the ambient noise at a location close to ear 186, by processing signals 188, 190 and 192.

It is further noted that since signals 188 and 190 are analog, two analog to digital converters (not shown), are employed to convert signals 188 and 190 to digital format. Alternatively, these analog to digital converters are integrated with each one of reference acoustoelectric transducer 174 and error acoustoelectric transducer 176, or integrated with digital ANR controller 180. Signal 192 can be either digital or analog. If signal 192 is analog, then another ADC (not shown) converts signal 192 to digital format. A digital to analog converter (not shown), and herein below referred to as DAC, converts signal 194 from digital format to analog format. Alternatively, this DAC is integrated with either digital ANR controller 180 or with primary summing element 184.

With further reference to FIG. 2B, analog ANR controller 182 includes a digital portion 228, an analog portion 230 and a secondary summing element 232. Secondary summing element 232 is coupled with digital portion 228, analog portion 230 and primary summing element 184. Primary summing element 184 is coupled with electroacoustic transducer 178. Analog portion 230 is coupled with error acoustoelectric transducer 176. Analog portion 230, primary summing element 184, secondary summing element 232, electroacoustic transducer 178 and error acoustoelectric transducer 176 form a feedback loop L₂ in system 170.

Following is a description of feedback loop L₂. Analog portion 230 receives signal 190 from error acoustoelectric transducer 176, produces a signal 234 and sends signal 234 to secondary summing element 232. Signal 234 is approximately 180 degrees out-of-phase relative to signal 190. Due to the operation of analog portion 230 and gain losses between electroacoustic transducer 178 and analog portion 230, signal 234 is attenuated. Digital portion 228 produces a signal 236 by attenuating signal 192 by the same amount that signal 234 is attenuated and sends signal 236 to secondary summing element 232.

Secondary summing element 232 produces a signal 198, by adding signals 234 and 236. Since the desired sound portion of signal 234 is out-of-phase by approximately 180 degrees relative to signal 236, the desired sound portion of signal 234 and signal 236, substantially cancel out at secondary summing element 232. Thus, signal 198 is substantially respective of only the anti-phase of the ambient noise at a location close to ear 186. Primary summing element 184 produces a signal 200 by adding signals 194 and 198. Electroacoustic transducer 178 emits a sound respective of the sum of signal 194 (which includes the desired sound, an anti-phase to the ambient noise at a location close to ear 186 and an adjustment according to signal 190) and signal 198 (which includes another anti-phase to the ambient noise at a location close to ear 186).

It is noted that the ANR controller can include only the digital ANR controller coupled with the reference acoustoelectric transducer, the error acoustoelectric transducer and with the electroacoustic transducer. Thus, the digital ANR controller makes adjustments to a signal which sends to the electroacoustic transducer, according to an error signal, which the digital ANR controller receives from the error acoustoelectric transducer. In this case, the digital ANR controller reduces mainly tonal noise.

With reference to FIG. 2A, it is noted that digital ANR controller 180 operates at a slower rate than that of analog ANR controller 182, but digital ANR controller 180 is substantially more effective in producing anti-phase signals for tonal noise and for noise at substantially high frequencies. On the other hand, analog ANR controller 182 is more effective in producing anti-phase signals for noise in a substantially wide frequency range, albeit at substantially low frequencies. Thus, by combining digital ANR controller 180 and analog ANR controller 182 in ANR controller 172, system 170 is capable to produce a desired sound in the presence of noise, both at a narrow (i.e., tonal noise) or a wide frequency range, as well as low or high frequencies. Digital ANR controller 180 and analog ANR controller 182 attenuate the same noise. Thus, the attenuated noise in signal 200 is substantially equal to the sum of the attenuation performed by digital ANR controller 180 and analog ANR controller 182.

With reference to FIG. 2C, system 170 includes ANR controller 202, reference acoustoelectric transducers 204 and 238, error acoustoelectric transducers 206 and 208 and electroacoustic transducers 210 and 212. ANR controller 202 is similar to ANR controller 172 (FIG. 2A). Each of error acoustoelectric transducers 206 and 208 is similar to error acoustoelectric transducer 176. Each of electroacoustic transducers 210 and 212 is similar to electroacoustic transducer 178. Error acoustoelectric transducers 206 and 208 and electroacoustic transducers 210 and 212 are coupled with head-mounted device 214. Reference acoustoelectric transducers 204 and 238 are located external to head-mounted device 214 or externally mounted thereon, but acoustically insulated or remote from error acoustoelectric transducers 206 and 208 and electroacoustic transducers 210 and 212.

Head-mounted device 214 is similar to head-mounted device 150, as described herein above in connection with FIG. 1C.

Error acoustoelectric transducer 206, electroacoustic transducer 210 and reference acoustoelectric transducer 238 are located adjacent to the right ear (not shown) of the user (not shown). Error acoustoelectric transducer 208, electroacoustic transducer 212 and reference acoustoelectric transducer 204 are located adjacent to the left ear (not shown) of the user. Error acoustoelectric transducer 206 detects the sound emitted by electroacoustic transducer 210, the ambient noise at a reduced SPL, and substantially none of the sound emitted by electroacoustic transducer 212. Error acoustoelectric transducer 208 detects the sound emitted by electroacoustic transducer 212, the ambient noise at a reduced SPL, and substantially none of the sound emitted by electroacoustic transducer 210. Reference acoustoelectric transducers 204 and 238 detect the ambient noise and substantially none of the sound which is emitted by electroacoustic transducers 210 and 212.

ANR controller 202 is coupled with reference acoustoelectric transducers 204 and 238, error acoustoelectric transducers 206 and 208 and with electroacoustic transducers 210 and 212. ANR controller 202 receives a signal 216 from reference acoustoelectric transducer 204, a signal 240 from reference acoustoelectric transducer 238, a signal 218 from error acoustoelectric transducer 206, a signal 220 from error acoustoelectric transducer 208 and a signal 222 from a sound source (not shown). Signals 216 and 238 are similar to signal 188 (FIG. 2A). Each of signals 218 and 220 is similar to signal 190. Each of signals 224 and 226 is similar to signal 200 and signal 222 is similar to signal 192.

Signal 222 can be either a single channel sound signal (i.e., monaural), or a multi-channel sound signal, such as stereophonic, quadraphonic, surround sound, and the like. ANR controller 202 produces a signal 224 for electroacoustic transducer 210 and a signal 226 for electroacoustic transducer 212. ANR controller 202 produces signals 224 and 226, by processing signals 216, 238, 218, 220 and 222, in the same manner that ANR controller 172 (FIG. 2A) processes signals 188, 192 and the signal received from error acoustoelectric transducer 176, for producing signal 200.

Each of electroacoustic transducers 210 and 212 produces a sound which includes the sound respective of signal 222 and an anti-phase of the ambient noise at a reduced SPL. Since the anti-phase of the ambient noise substantially cancels the actual ambient noise at the quiet zone of the respective ear, the user hears mostly a sound corresponding to signal 222 and substantially none of the ambient noise. If signal 222 is a single channel sound signal, then each of signals 224 and 226 is produced according to signal 222 and the anti-phase of the ambient noise at a reduced SPL. Hence, the user can hear a monaural sound.

If signal 222 is stereo, then signals 224 and 226 are produced for example, according to the right and the left channel of signal 222, respectively, and according to the anti-phase of the ambient noise at a reduced SPL. Hence, the user can hear the sound which corresponds to signal 222 in stereo, without hearing the ambient noise.

Alternatively, more than two electroacoustic transducers and respective acoustoelectric transducers can be coupled to the ANR controller. In this case, if signal 222 is multi-channel, then the user can hear the sound which corresponds to signal 222 in multi-dimension, without hearing the ambient noise.

With further reference to FIG. 2A, the electroacoustic transducers are coupled with the primary summing element and the acoustoelectric transducers are coupled with the digital ANR controller. The digital ANR controller produces a signal for each one of the electroacoustic transducers, by processing the desired sound signal, the noise signal and the error signal received from the respective acoustoelectric transducer.

With further reference to FIG. 2B, the electroacoustic transducers are coupled with the primary summing element and the acoustoelectric transducers are coupled with the analog portion of the analog ANR controller. According to the desired sound signal, the digital portion estimates in real time, the SPL of the desired sound which each of the electroacoustic transducers produces and the digital portion produces these estimated desired sound signals. The digital portion sends the estimated desired sound signal respective of each of the electroacoustic transducers, to the secondary summing element.

The analog portion produces an anti-phase signal respective of each of the signals received from the acoustoelectric transducers and sends these anti-phase signals to the secondary summing element. The secondary summing element produces a signal respective of each of the electroacoustic transducers, by adding the respective anti-phase signal received from the analog portion and the respective signal received from the digital portion. The primary summing element produces a signal for each of the electroacoustic transducers, by adding the respective signal received from the digital ANR controller and the respective signal received from the secondary summing element.

Alternatively, the noise-canceling system of FIG. 2A, receives no signals respective of the desired sound and produces only an anti-phase noise sound, according to noise detected by a reference acoustoelectric transducer located away from the ear of the user. In this case, the noise-canceling system includes a digital ANR controller similar to digital ANR controller 180, a reference acoustoelectric transducer and an electroacoustic transducer. The digital ANR controller is coupled with the reference acoustoelectric transducer and the electroacoustic transducer. The reference acoustoelectric transducer is located in a noisy environment away from the ear of the user and the electroacoustic transducer is located close to the ear of the user.

Additionally, the noise-canceling system includes an error acoustoelectric transducer coupled with the digital ANR controller. The error acoustoelectric transducer is located close to the ear of the user and sends an error signal to the digital ANR controller, respective of the sound emitted by the electroacoustic transducer. The digital ANR controller processes the error signal and the reference noise signal and makes adjustments to the anti-phase noise signal which sends to the electroacoustic transducer.

Additionally, the noise-canceling system includes an analog ANR controller similar to analog ANR controller 182 and a summing element. The analog ANR controller is coupled with the error acoustoelectric transducer and the summing element, and the summing element is coupled with the digital ANR controller and the electroacoustic transducer. The analog ANR controller produces an anti-phase noise signal approximately 180 degrees out-of-phase relative to the error signal. The summing element produces a signal for the electroacoustic transducer, by adding the anti-phase noise signals produced by the digital ANR controller and the analog ANR controller.

Alternatively, the error acoustoelectric transducer can be coupled only with the analog active noise reduction controller and not with the digital active noise reduction controller. In this case, only the analog active noise reduction controller makes adjustments to the anti-phase noise signal which the digital active noise reduction controller sends to the electroacoustic transducer.

According to another aspect of the disclosed technique, a noise reduction system produces a noise-free sound close to the ear of a user, and a noise-free signal corresponding to the voice of the user. The system produces a noise-canceling sound or a noise canceling signal, according to a noise reference signal.

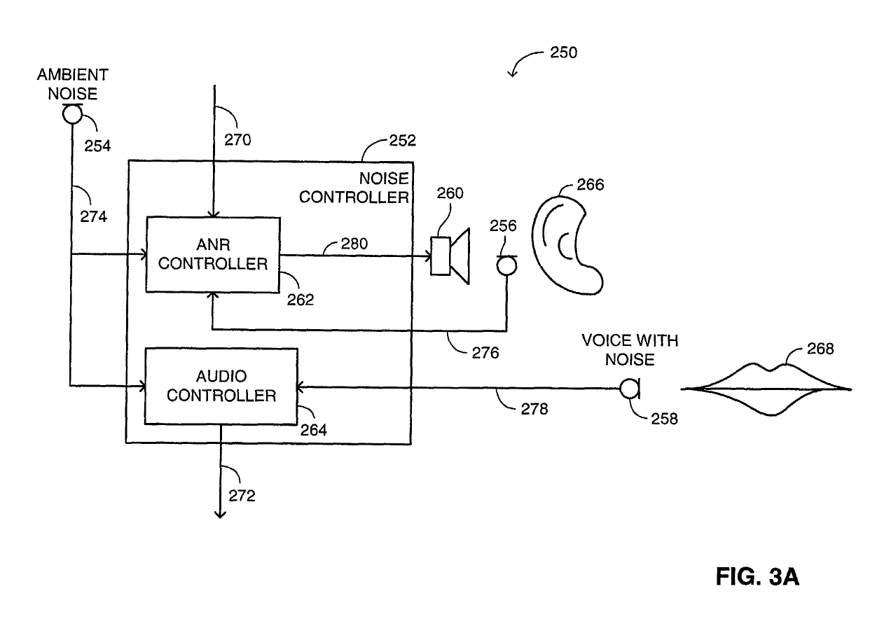

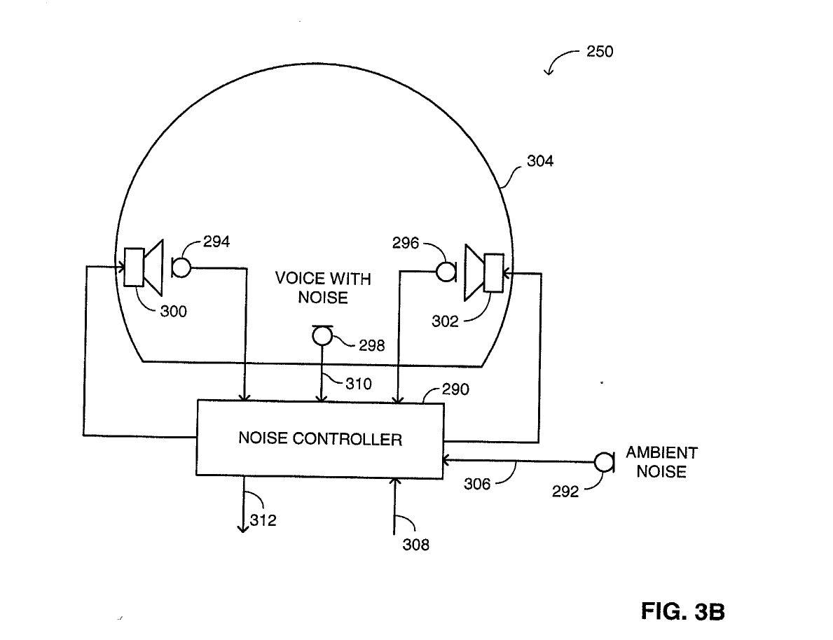

Reference is now made to FIGS. 3A and 3B. FIG. 3A is a schematic illustration of a noise reduction system, generally referenced 250, constructed and operative in accordance with a further embodiment of the disclosed technique. FIG. 3B is a schematic illustration of the system of FIG. 3A, incorporated with a head-mounted device, generally referenced 304.

With reference to FIG. 3A, system 250 includes a noise controller 252, a reference acoustoelectric transducer 254, an error acoustoelectric transducer 256, a voice acoustoelectric transducer 258 and an electroacoustic transducer 260. Noise controller 252 includes an ANR controller 262 and an audio controller 264. ANR controller 262 is similar to ANR controller 172 (FIG. 2A) and audio controller 264 is similar to audio controller 106 (FIG. 1A).

ANR controller 262 is coupled with reference acoustoelectric transducer 254, error acoustoelectric transducer 256 and with electroacoustic transducer 260. Audio controller 264 is coupled with reference acoustoelectric transducer 254 and voice acoustoelectric transducer 258.

Electroacoustic transducer 260 and error acoustoelectric transducer 256 are located close to an ear 266 of a user (not shown) and voice acoustoelectric transducer 258 is located close to a mouth 268 of the user. Sound absorbing material (not shown) can be placed between electroacoustic transducer 260, error acoustoelectric transducer 256 and voice acoustoelectric transducer 258 on one side and reference acoustoelectric transducer 254, on the other. Such a sound absorbing material can be in the form of an earmuff, and the like, which encloses electroacoustic transducer 260 and error acoustoelectric transducer 256. In addition, sound absorbing material acoustically insulates voice acoustoelectric transducer 258 and mouth 268 from electroacoustic transducer 260, error acoustoelectric transducer 256 and ear 266. Thus, error acoustoelectric transducer 256 does not detect the voice of the user and voice acoustoelectric transducer 258 does not detect sound emitted by electroacoustic transducer 260.

Thus, reference acoustoelectric transducer 254 detects the ambient noise and substantially none of the voice of the user or the sound emitted by electroacoustic transducer 260. Reference acoustoelectric transducer 254 sends a signal 274 respective of the detected ambient noise, to ANR controller 262 and to audio controller 264. Error acoustoelectric transducer 256 detects the sound emitted by electroacoustic transducer 260 and the ambient noise at a reduced SPL and sends a respective signal 276 to ANR controller 262. Voice acoustoelectric transducer 258 detects the voice of the user from mouth 268 and the ambient noise at a reduced SPL and sends a respective signal 278 to audio controller 264.

System 250 can be divided to a hearing portion and a speaking portion. The hearing portion consists of ANR controller 262, reference acoustoelectric transducer 254, error acoustoelectric transducer 256 and electroacoustic transducer 260. The speaking portion consists of audio controller 264 and reference acoustoelectric transducer 254 and voice acoustoelectric transducer 258. Reference acoustoelectric transducer 254 is common to the hearing portion and the speaking portion.

The hearing portion of system 250 is similar to system 170, as described herein above in connection with FIG. 2A. ANR controller 262 determines an anti-phase to signal 274 at a reduced SPL (i.e., the ambient noise at the quiet zone of ear 266). ANR controller 262 produces a signal 280 respective of the desired sound, according to a signal 270 from a sound source (not shown) and the anti-phase of signal 274 at the reduced SPL. Electroacoustic transducer 260 produces a sound according to signal 280. Thus, the user hears the desired sound and substantially none of the ambient noise. ANR controller 262 makes adjustments to signal 280, according to signal 276.

Alternatively, the active noise reduction controller does not receive any signal respective of the desired sound. In this case, the active noise reduction controller sends a noise-canceling signal to the electroacoustic transducer and a is different electroacoustic transducer produces the desired sound according to the signal respective of the desired sound. Further alternatively, the desired sound reaches the ear from a sound source other than an electroacoustic transducer, such as the voice of another person, mechanical voice, machine generated sound, and the like.

Alternatively, the acoustoelectric transducer can be eliminated from the noise reduction system. In this case, the active noise reduction controller produces a noise-canceling signal only according to the reference noise signal, and without any error signal as feedback.

The speaking portion of system 250 is similar to system 100, as described herein above in connection with FIG. 1A. Thus, audio controller 264 produces a noise-free voice signal 272.

With reference to FIG. 3B, system 250 includes a noise controller 290, a reference acoustoelectric transducer 292, error acoustoelectric transducers 294 and 296, a voice acoustoelectric transducer 298 and electroacoustic transducers 300 and 302. Noise reduction system 290 is similar to noise reduction system 252 (FIG. 3A). Noise controller 290 is coupled with reference acoustoelectric transducer 292, error acoustoelectric transducers 294 and 296, voice acoustoelectric transducer 298 and with electroacoustic transducers 300 and 302.

Error acoustoelectric transducers 294 and 296, voice acoustoelectric transducer 298 and electroacoustic transducers 300 and 302 are located within head-mounted device 304. Reference acoustoelectric transducer 292 is located external to head-mounted device 304 or externally mounted thereon, but acoustically insulated or remote the mouth of the user and from error acoustoelectric transducers 294 and 296 and electroacoustic transducers 300 and 302. Error acoustoelectric transducer 294 and electroacoustic transducer 300 are located at a right ear (not shown) of a user (not shown). Error acoustoelectric transducer 296 and electroacoustic transducer 302 are located at a left ear (not shown) of the user. Voice acoustoelectric transducer 298 is located at a mouth (not shown) of the user.

Noise controller 290 receives a signal 306 from reference acoustoelectric transducer 292, respective of the ambient noise and a signal 308 from a sound source (not shown), respective of a desired sound. Noise controller 290 receives a signal 310 from voice acoustoelectric transducer 298 respective of the voice of the user and the ambient noise at a reduced SPL.

Noise controller 290, reference acoustoelectric transducer 292, error acoustoelectric transducers 294 and 296 and electroacoustic transducers 300 and 302, form the hearing portion of system 250, as described herein above in connection with FIG. 3A. Electroacoustic transducers 300 and 302 produce sounds which include a desired sound carried by a signal 308 and another sound at anti-phase and at a reduced SPL relative to signal 306. Thus, the user hears the desired sound and substantially none of the ambient noise.

Noise controller 290, reference acoustoelectric transducer 292 and voice acoustoelectric transducer 298, form the speaking portion of system 250, as described herein above in connection with FIG. 2A. Thus, noise controller 290 produces a noise-free signal 312 of the voice of the user, according to signals 306 and 310.

Alternatively, system 250 can include two reference acoustoelectric transducers similar to reference acoustoelectric transducer 292 and coupled with noise controller 290. Each of these reference acoustoelectric transducers is located external to head-mounted device 304, in a manner similar to that described herein above in connection with reference acoustoelectric transducers 204 and 238 (FIG. 2C).

According to another aspect of the disclosed technique, an active noise reduction system includes a digital feedforward portion which receives a reference noise signal and a digital/analog feedback portion, which receives a signal respective of a sound produced by the system at the quiet zone of the ear. The feedforward portion produces a signal respective of a desired sound, and an anti-phase of the background noise according to a desired sound signal and the feedback from the feedback portion.

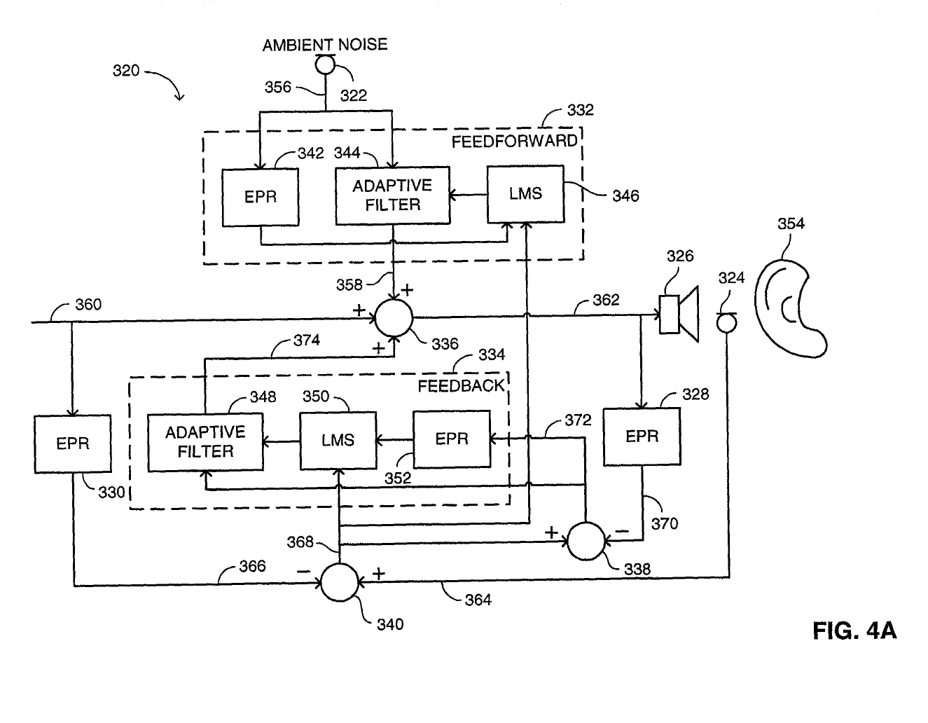

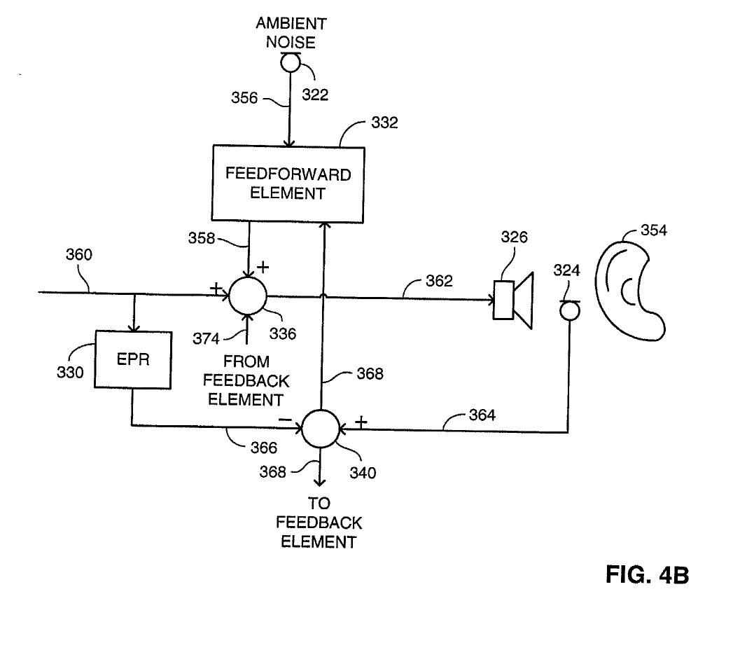

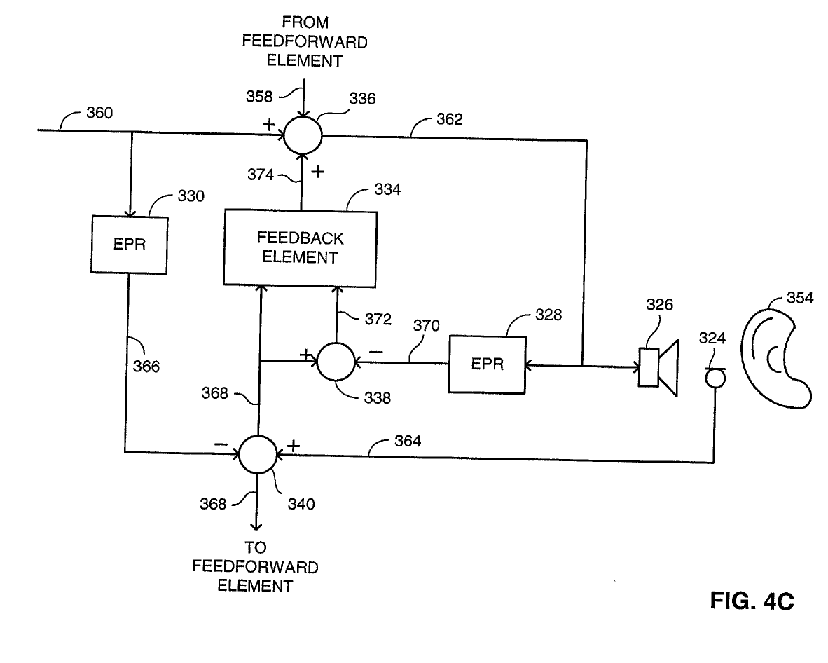

Reference is now made to FIGS. 4A, 4B and 4C. FIG. 4A is a schematic illustration of a digital noise reduction system, generally referenced 320, constructed and operative in accordance with another embodiment of the disclosed technique. FIG. 4B is a schematic illustration of the feedforward portion of the system of FIG. 4A. FIG. 4C is a schematic illustration of the feedback portion of the system of FIG. 4A. It is noted that system 320 is a detail illustration of a digital ANR controller such as digital ANR controller 180 (FIG. 2A).

With reference to FIG. 4A, system 320 includes a reference acoustoelectric transducer 322, an error acoustoelectric transducer 324, an electroacoustic transducer 326, estimated plant response (EPR) elements 328 and 330, a feedforward element 332, a feedback element 334, and summing elements 336, 338 and 340. Feedforward element 332, feedback element 334, EPR elements 328 and 330 and summing elements 336, 338 and 340 together, are equivalent to digital ANR controller 180 (FIG. 2A). Feedforward element 332 includes an EPR element 342, an adaptive filter 344 and a least mean square (LMS) element 346. Feedback element 334 includes an adaptive filter 348, an LMS element 350 and an EPR element 352.

An EPR element is an element which estimates the ratio of two sound signals according to predetermined information, applies this ratio to an input signal to the EPR element and produces an output signal, accordingly. One of these two sound signals can be for example, respective of a desired sound which is to be produced by an electroacoustic transducer, while the other sound signal is respective of the sound which the electroacoustic transducer actually produces. An LMS element is an element which updates the response of the adaptive filter, according to an LMS adaptive filter method. The combination of an LMS element and an EPR element is equivalent to a Filter X LMS (FXLMS) element, as known in the art.

Electroacoustic transducer 326 and error acoustoelectric transducer 324 are located close to an ear 354 of a user (not shown). A sound absorbing element (not shown) is located between electroacoustic transducer 326 and error acoustoelectric transducer 324 on one side and reference acoustoelectric transducer 322 on the other. Thus, reference acoustoelectric transducer 322 detects the ambient noise and none of the sound emitted by electroacoustic transducer 326. Error acoustoelectric transducer 324 detects the sound emitted by electroacoustic transducer 326 and the ambient noise at a reduced SPL. Each of adaptive filters 344 and 348 is similar in principle to adaptive filter 118, as described herein above in connection with FIG. 1B.

With reference to FIG. 4B, the digital feedforward portion of system 320 includes reference acoustoelectric transducer 322, error acoustoelectric transducer 324, electroacoustic transducer 326, feedforward element 332, summing elements 336 and 340 and EPR element 330. Summing element 336 is coupled with feedforward element 332, electroacoustic transducer 326 and with EPR element 330. Summing element 340 is coupled with feedforward element 332, error acoustoelectric transducer 324 and with EPR element 330. Reference acoustoelectric transducer 322 is coupled with feedforward element 332.

Reference acoustoelectric transducer 322 detects the ambient noise and sends a respective signal 356 to feedforward element 332. Feedforward element 332 determines the reduced SPL of the ambient noise at the quiet zone of ear 354. It is noted that the SPL reduction is generally sensitive to the frequency of signal 356. Feedforward element 332, determines a signal 358 which is at anti-phase to the ambient noise signal 356 at the reduced SPL and sends signal 358 to summing element 336. Summing element 336 adds signal 358 to a signal 360, and produces a signal 362 respective of the result of addition. Signal 360 is respective of a desired sound from a sound source (not shown). Thus, signal 362 includes the desired sound signal and the anti-phase of the ambient noise at the reduced SPL. Summing element 336 sends signal 362 to electroacoustic transducer 326.

Electroacoustic transducer 326 produces the desired sound together with a noise-canceling sound, according to signal 362. Since the anti-phase of the ambient noise at the quiet zone of ear 354 cancels the ambient noise at this quiet zone, the user hears the desired sound and substantially none of the ambient noise.

Error acoustoelectric transducer 324 detects the sound emitted by electroacoustic transducer 326 and sends a signal 364 respective of the detected sound, to summing element 340. EPR element 330 receives signal 360, determines a signal 366 which is an estimate of the desired sound emitted by electroacoustic transducer 326 at the quiet zone of ear 354, and sends signal 366 to summing element 340. Summing element 340 produces an error signal 368, by comparing signals 366 and 364 (i.e., by subtracting signal 366 from signal 364) and sends error signal 368 to feedforward element 332 and to feedback element 334. Error signal 368 represents the difference between the desired sound as received from the sound source and the noise-cancelled desired sound emitted at the quiet zone of ear 354. Feedforward element 332 makes a correction to signal 358 according to error signal 368 and sends signal 358 to summing element 336.

With reference to FIG. 4C, the feedback portion of system 320 includes electroacoustic transducer 326, error acoustoelectric transducer 324, feedback element 334, EPR elements 328 and 330 and summing elements 336, 338 and 340. Summing element 336 is coupled with feedback element 334, EPR elements 328 and 330 and with electroacoustic transducer 326. Summing element 338 is coupled with feedback element 334, EPR element 328 and with summing element 340. Summing element 340 is coupled with feedback element 334, EPR element 330, summing element 338 and with error acoustoelectric transducer 324.

Summing element 336 produces signal 362 by adding signal 358, which summing element 336 receives from feedforward element 332, to signal 360, which summing element 336 receives from the sound source. Thus, as described herein above in connection with FIG. 4B, signal 362 includes the desired sound signal and the anti-phase of the ambient noise at the reduced SPL. Summing element 336 sends signal 362 to electroacoustic transducer 326 and to EPR element 328.

Electroacoustic transducer 326 produces the desired sound together with a noise-canceling sound, according to signal 362. Since the anti-phase of the ambient noise at the quiet zone of ear 354 cancels the ambient noise at this quiet zone, the user hears the desired sound and substantially none of the ambient noise.

Error acoustoelectric transducer 324 detects the sound emitted by electroacoustic transducer 326 and sends a signal 364 respective of the detected sound, to summing element 340. EPR element 330 receives signal 360, determines a signal 366 which is an estimate of the desired sound emitted at the quiet zone of ear 354 and sends signal 366 to summing element 340. Summing element 340 produces an error signal 368, by comparing signals 366 and 364 (i.e., by subtracting signal 366 from signal 364) and sends error signal 368 to feedback element 334, to summing element 338 and to feedforward element 332. Error signal 368 represents the difference between the desired sound as received from the sound source and the noise-cancelled desired sound emitted at the quiet zone of ear 354.

EPR element 328 produces a signal 370, which is an estimate of a sound emitted by electroacoustic transducer 326 and as detected by error acoustoelectric transducer 324. EPR element 328 produces signal 370 according to signal 362. Summing element 338 produces an error signal 372, by comparing signals 368 and 370 (i.e., by subtracting signal 370 from signal 368) and sends error signal 372 to feedback element 334. Feedback element 334 produces an error signal 374, by processing error signals 368 and 372 and sends error signal 374 to summing element 336. Summing element 336 produces signal 362 by adding error signal 374 to signal 358 (for the ambient noise canceling signal) and signal 360 (for the sound source signal).

It is noted that the noise reduction system can include a plurality of electroacoustic transducers and a respective acoustoelectric transducer for each of the electroacoustic transducers. In this case, the system receives the desired sound in a plurality of channels and the user can hear the desired sound in multiple dimensions.

It is further noted that system 320 produces an anti-phase noise signal according to a signal received from an acoustoelectric transducer (i.e., reference acoustoelectric transducer 322), which is not affected by the sound emitted by the electroacoustic transducer (i.e., electroacoustic transducer 326) and adapts this anti-phase noise signal according to a signal respective of the sound emitted by this electroacoustic transducer (i.e., signal 364). The operation of the feedforward portion and the feedback portion of system 320 are similar. The difference between the two portions is that the input to the feedforward portion is the ambient noise devoid of any sound emitted by the electroacoustic transducer, while the input to the feedback portion is the sound which is actually emitted by this electroacoustic transducer.

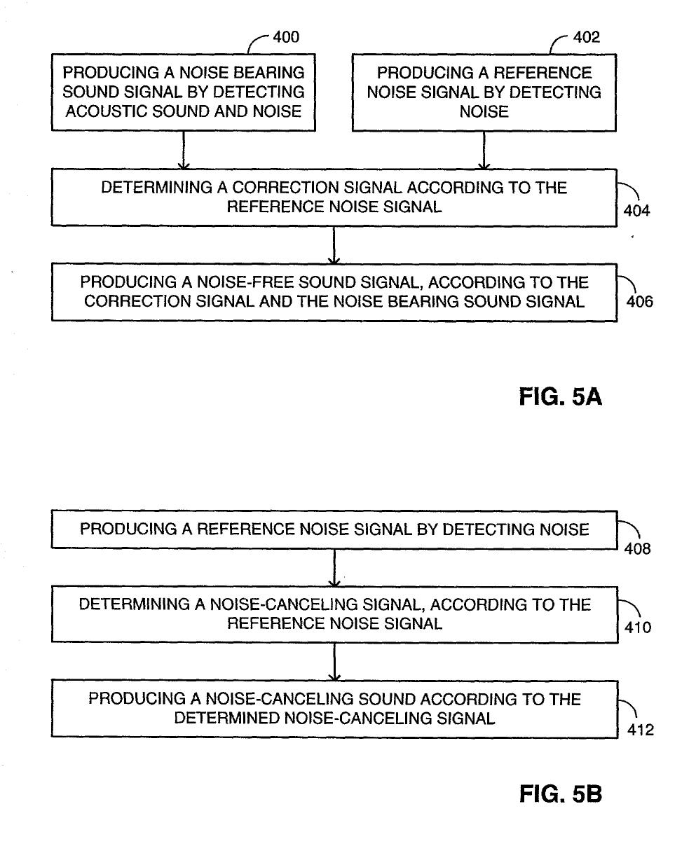

Reference is now made to FIG. 5A, which is a schematic illustration of a method for operating the system of FIG. 1A, operative in accordance with a further embodiment of the disclosed technique. In procedure 400 a noise bearing sound signal is produced, by detecting acoustic sound and noise. With reference to FIG. 1A, acoustoelectric transducer 102 detects acoustic sound and noise and sends signal 108 respective of this detected acoustic sound and noise, to audio controller 106.

In procedure 402, a reference noise signal is produced by detecting noise. With reference to FIG. 1A, acoustoelectric transducer 104 detects the noise and sends signal 110 respective of this noise, to audio controller 106.

In procedure 404, a correction signal is determined according to the reference noise signal. With reference to FIG. 1A, audio controller 106 determines a reduced SPL for signal 110.

In procedure 406, a noise-free signal is produced according to the correction signal and the noise bearing sound signal. With reference to FIG. 1A, audio controller 106 produces signal 112 by subtracting signal 110 at the reduced SPL, from signal 108.

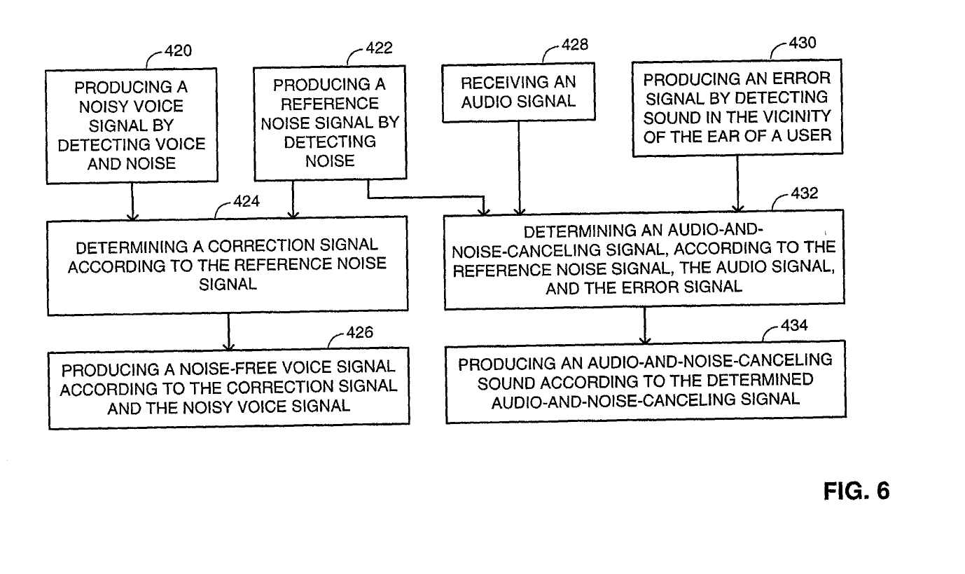

Reference is now made to FIG. 5B, which is a schematic illustration of a method for operating a noise-canceling system, operative in accordance with another embodiment of the disclosed technique. This noise-canceling system employs a reference acoustoelectric transducer to detect the ambient noise, wherein the reference acoustoelectric transducer is located away from the ear of the user. It is noted that the procedure of detecting the ambient noise by this reference acoustoelectric transducer, is common to both of the methods according to FIGS. 5A and 5B. It is further noted that the methods according to FIGS. 5A and 5B, can be combined into a single method which is herein below described in connection with FIG. 6.

With reference to FIG. 5B, in procedure 408, which is similar to procedure 402, reference noise signal is produced by detecting noise. The reference acoustoelectric transducer produces a reference noise signal, by detecting the ambient noise. In procedure 410, a noise-canceling signal is determined, by processing the reference noise signal. An ANR controller similar to ANR controller 172 (FIG. 2A) determines a noise-canceling signal by processing the reference noise signal. The ANR controller determines a reduced SPL for the reference noise signal, corresponding to the SPL of the ambient noise at a location close to the ear of the user. Furthermore, the ANR controller determines a noise-canceling signal, which is approximately 180 degrees out-of-phase relative to the reference noise signal. An electroacoustic transducer similar to electroacoustic transducer 178 (FIG. 2A), produces a noise-canceling sound according to the determined noise-canceling signal (procedure 412).

Reference is now made to FIG. 6, which is a schematic illustration of a method for operating the system of FIG. 3A, operative in accordance with a further embodiment of the disclosed technique. In procedure 420, a noisy voice signal is produced by detecting voice and noise. With reference to FIG. 3A, voice acoustoelectric transducer 258 detects the voice of the user from mouth 268, together with the ambient noise at a reduced SPL and sends signal 278 to audio controller 264.

In procedure 422, a reference noise signal is produced by detecting noise. With reference to FIG. 3A, reference acoustoelectric transducer 254 detects the ambient noise and sends signal 274 to audio controller 264.

In procedure 424, a correction signal is determined according to the reference noise signal. With reference to FIG. 3A, audio controller 264 determines a reduced SPL for signal 274.

In procedure 426, a noise-free voice signal is produced according to the correction signal and the noisy voice signal. With reference to FIG. 3A, audio controller 264 produces signal 272 by subtracting signal 274 at the reduced SPL, from signal 278.

In procedure 428, an audio signal is received. With reference to FIG. 3A, ANR controller 262 receives signal 270 from the sound source. In procedure 430, an error signal is produced, by detecting sound in the vicinity of the ear. With reference to FIG. 3A, error acoustoelectric transducer 256 detects the sound close to ear 266 and sends signal 276 respective of this detected sound, to ANR controller 262.

In procedure 432, an audio-and-noise-canceling signal is determined, according to the reference noise signal, the audio signal and the error signal. With reference to FIG. 3A, ANR controller 262 determines signal 280, by processing signals 270, 274 and 276.

In procedure 434, an audio-and-noise-canceling sound is produced according to the determined audio-and-noise-canceling signal. With reference to FIG. 3A, electroacoustic transducer 260 produces sound according to signal 280.

It will be appreciated by persons skilled in the art that the disclosed technique is not limited to what has been particularly shown and described hereinabove. Rather the scope of the disclosed technique is defined only by the claims, which follow.

1. System for producing a substantially noise-free signal of an acoustic sound, and for producing at least one sound, the at least one sound including a desired sound and an anti-phase noise sound, the anti-phase noise sound being in anti-phase relative to a noise, the system comprising:
an acoustoelectric transducer for producing a noise bearing sound signal by detecting said acoustic sound and said noise;
a reference-acoustoelectric transducer for producing a reference noise signal by detecting said noise in a noisy environment; and
an audio controller coupled with said reference-acoustoelectric transducer and said acoustoelectric transducer,
wherein said audio controller produces said substantially noise-free signal, according to said reference noise signal and said noise bearing sound signal.

2. The system according to claim 1, wherein said acoustic sound is a voice of a user talking in said noisy environment,
wherein said acoustoelectric transducer is a voice-acoustoelectric transducer, and
wherein said noise bearing sound signal is a noisy voice signal.

3. The system according to claim 1, wherein said audio controller produces said substantially noise-free signal, by determining a reduced-intensity noise signal according to said reference noise signal, and by subtracting said determined reduced-intensity noise signal from said noise bearing sound signal,
wherein said determined reduced-intensity noise signal corresponds with the intensity of said noise at a location substantially close to said acoustoelectric transducer.

4. The system according to claim 1, wherein said audio controller employs a sound pressure level converter selected from the list consisting of:
look-up table; and
transfer function.

5. The system according to claim 2, wherein said audio controller employs a sound pressure level converter, and wherein the form and conversion parameters of said sound pressure level converter are determined according to at least one physical characteristic selected from the list consisting of:
hearing characteristics of another user;
voice characteristics of said user;
sound absorbing characteristics of a headset worn by said user;
distance between said reference-acoustoelectric transducer and said voice-acoustoelectric transducer;
acoustic properties of the environment which surrounds said reference-acoustoelectric transducer and said voice-acoustoelectric transducer; and
acoustic properties of said reference-acoustoelectric transducer and said voice-acoustoelectric transducer.

6. The system according to claim 1, wherein said reference-acoustoelectric transducer and said acoustoelectric transducer are acoustically separated.

7. The system according to claim 1, wherein each of said reference-acoustoelectric transducer and said acoustoelectric transducer operates according to a principle selected from the list consisting of:
electrodynamics;
electrostatics;
piezoelectricity;
magnetostriction;
fiber-optics; and
stimulation of carbon particles.

8. The system according to claim 1, wherein the source of said noise is selected from the list consisting of:
at least one person;
engine;
turbine;
motor;
mechanical device;
hydraulic device;
pneumatic device;
electromechanical device;
loud-speaker;
firing of ammunition;
environment;
geological source; and
animal.

9. The system according to claim 1, further including a first analog to digital converter, for converting said reference noise signal from analog to digital.

10. The system according to claim 1, further including a second analog to digital converter, for converting said noise bearing sound signal from analog to digital.

11. The system according to claim 1, wherein said audio controller includes:
an adaptive filter coupled with said reference-acoustoelectric transducer, and
a summing element coupled with said adaptive filter and with said acoustoelectric transducer,
wherein said adaptive filter determines a reduced-intensity noise signal according to said reference noise signal,
wherein said summing element produces said substantially noise-free signal by subtracting said reduced-intensity noise signal from said noise bearing sound signal, and
wherein said summing element feeds back said substantially noise-free signal to said adaptive filter.

12. The system according to claim 1, wherein said audio controller is wirelessly coupled with said reference-acoustoelectric transducer.

13. The system according to claim 1, wherein said audio controller is wirelessly coupled with said acoustoelectric transducer.

14. The system according to claim 1, wherein said acoustoelectric transducer and said reference-acoustoelectric transducer are coupled with a head-mounted device.

15. The system according to claim 14, wherein the type of said head-mounted device is selected from the list consisting of:
helmet; and
headset.

16. The system according to claim 14, wherein said head-mounted device includes a sound absorbing material, and
wherein said sound absorbing material acoustically separates between said reference-acoustoelectric transducer and said acoustoelectric transducer.

17. The system according to claim 14, wherein said head-mounted device includes a visual device.

18. The system according to claim 17, wherein the type of said visual device is selected from the list consisting of:
head-up display;
visor;
liquid crystal display;
field emission display; and
mirror.

19. The system according to claim 1, further including:
at least one electroacoustic transducer for producing said at least one sound; and
an active noise reduction controller coupled with said at least one electroacoustic transducer and said reference-acoustoelectric transducer,
wherein said active noise reduction controller produces at least one sound signal according to said reference noise signal and according to a desired sound signal respective of said desired sound, and
wherein said at least one electroacoustic transducer produces said at least one sound, according to a respective one of said at least one sound signal.

20. The system according to claim 19, wherein said active noise reduction controller is wirelessly coupled with said reference-acoustoelectric transducer.

21. The system according to claim 19, wherein said active noise reduction controller is wirelessly coupled with said at least one electroacoustic transducer.

22. The system according to claim 19, further including at least one error-acoustoelectric transducer coupled with said active noise reduction controller,
wherein said at least one error-acoustoelectric transducer produces at least one error signal, by detecting a respective one of said at least one sound,
wherein said active noise reduction controller produces a first anti-phase noise signal respective of said at least one electroacoustic transducer, according to said at least one error signal, said desired sound signal and said reference noise signal, and
wherein said at least one electroacoustic transducer produces a respective new anti-phase noise sound according to said first respective anti-phase noise signal.

23. The system according to claim 22, wherein said active noise reduction controller is wirelessly coupled with said at least one error-acoustoelectric transducer.

24. The system according to claim 22, wherein said at least one electroacoustic transducer and a respective one of said at least one error-acoustoelectric transducer, are acoustically separated from said reference-acoustoelectric transducer.

25. The system according to claim 22, wherein said active noise reduction controller includes a digital active noise reduction controller coupled with said reference-acoustoelectric transducer, said at least one electroacoustic transducer and with said at least one error-acoustoelectric transducer,
wherein said digital active noise reduction controller produces a second anti-phase noise signal respective of said at least one electroacoustic transducer, according to said at least one error signal, said desired sound signal and said reference noise signal, and
wherein said at least one electroacoustic transducer produces said new respective anti-phase noise sound according to said second respective anti-phase noise signal.

26. The system according to claim 25, wherein said active noise reduction controller further includes:
an analog active noise reduction controller coupled with said at least one error-acoustoelectric transducer; and
a first summing element coupled with said digital active noise reduction controller, said analog active noise reduction controller and with said at least one electroacoustic transducer,
wherein said analog active noise reduction controller produces a third anti-phase noise signal respective of said at least one electroacoustic transducer, according to said at least one error signal and said desired sound signal,
wherein said first summing element produces said first respective anti-phase noise signal, by adding said second respective anti-phase noise signal and said third respective anti-phase noise signal, and
wherein said at least one electroacoustic transducer produces said new respective anti-phase noise sound according to said first respective anti-phase noise signal.

27. The system according to claim 26, wherein said analog active noise reduction controller includes:
a digital portion;
a second summing element coupled with said digital portion and said first summing element; and
an analog portion coupled with said second summing element and said at least one error-acoustoelectric transducer,
wherein said digital portion produces an estimated desired sound signal, respective of said desired sound as produced by said at least one electroacoustic transducer,
wherein said analog portion produces an anti-phase signal respective of said at least one error signal, and
wherein said second summing element produces said third respective anti-phase noise signal, by adding said respective estimated desired sound signal and said respective anti-phase signal.

28. The system according to claim 22, wherein said at least one electroacoustic transducer, said at least one error-acoustoelectric transducer and said reference-acoustoelectric transducer are coupled with a head-mounted device.

29. The system according to claim 19, wherein said active noise reduction controller receives said desired sound signal in at least one channel.

30. The system according to claim 1, wherein said desired sound is selected from the list consisting of:
human voice;
machine generated sound;
mechanical voice;
sound signal; and
acoustic sound.

31. System for producing at least one sound, the at least one sound including a desired sound and an anti-phase noise sound, the anti-phase noise sound being in anti-phase relative to a noise, the system comprising:
at least one electroacoustic transducer for producing said at least one sound;
at least one reference-acoustoelectric transducer for producing at least one reference noise signal by detecting said noise in a noisy environment; and
an active noise reduction controller coupled with said at least one electroacoustic transducer and said at least one reference-acoustoelectric transducer,
wherein said active noise reduction controller produces at least one sound signal according to said at least one reference noise signal and according to a desired sound signal respective of said desired sound, and
wherein said at least one electroacoustic transducer produces said at least one sound, according to a respective one of said at least one sound signal.

32. The system according to claim 31, further comprising:
an audio controller coupled with said reference-acoustoelectric transducer and with a acoustoelectric transducer, said acoustoelectric transducer producing a noise bearing sound signal by detecting an acoustic sound and said noise, and
wherein said audio controller produces a substantially noise-free signal of said acoustic sound, according to said reference noise signal and said noise bearing sound signal.

33. The system according to claim 31, further including at least one error-acoustoelectric transducer coupled with said active noise reduction controller,
wherein said at least one error-acoustoelectric transducer produces at least one error signal, by detecting a respective one of said at least one sound,
wherein said active noise reduction controller produces a first anti-phase noise signal respective of said at least one electroacoustic transducer, according to said at least one error signal, said desired sound signal and said at least one reference noise signal, and
wherein said at least one electroacoustic transducer produces a respective new anti-phase noise sound according to said first respective anti-phase noise signal.

34. The system according to claim 33, wherein said active noise reduction controller comprises a digital active noise reduction controller coupled with said at least one reference-acoustoelectric transducer, said at least one electroacoustic transducer and with said at least one error-acoustoelectric transducer,
wherein said digital active noise reduction controller produces a second anti-phase noise signal respective of said at least one electroacoustic transducer, according to said at least one error signal, said desired sound signal and said at least one reference noise signal, and
wherein said at least one electroacoustic transducer produces said new respective anti-phase noise sound according to said second respective anti-phase noise signal.

35. The system according to claim 34, wherein said active noise reduction controller further comprises:
an analog active noise reduction controller coupled with said at least one error-acoustoelectric transducer; and
a first summing element coupled with said digital active noise reduction controller, said analog active noise reduction controller and with said at least one electroacoustic transducer,
wherein said analog active noise reduction controller produces a third anti-phase noise signal respective of said at least one electroacoustic transducer, according to said at least one error signal and said desired sound signal,
wherein said first summing element produces said first respective anti-phase noise signal, by adding said second respective anti-phase noise signal and said third respective anti-phase noise signal, and
wherein said at least one electroacoustic transducer produces said new respective anti-phase noise sound according to said first respective anti-phase noise signal.

36. The system according to claim 35, wherein said analog active noise reduction controller comprises:
a digital portion;
a second summing element coupled with said digital portion and said first summing element; and
an analog portion coupled with said second summing element and said at least one error-acoustoelectric transducer,
wherein said digital portion produces an estimated desired sound signal, respective of said desired sound as produced by said at least one electroacoustic transducer,
wherein said analog portion produces an anti-phase signal respective of said at least one error signal, and
wherein said second summing element produces said third respective anti-phase noise signal, by adding said respective estimated desired sound signal and said respective anti-phase signal.

37. The system according to claim 33, wherein said at least one electroacoustic transducer and a respective one of said at least one error-acoustoelectric transducer, are acoustically separated from said at least one reference-acoustoelectric transducer.

38. System for producing an anti-phase noise sound, the system comprising:
an electroacoustic transducer;
a reference-acoustoelectric transducer for producing a reference noise signal by detecting noise in a noisy environment; and
a digital active noise reduction controller coupled with said electroacoustic transducer and said reference-acoustoelectric transducer,
wherein said digital active noise reduction controller produces a first anti-phase noise signal according to said reference noise signal, said first anti-phase noise signal being in anti-phase relative to said reference noise signal, and
wherein said electroacoustic transducer produces said anti-phase noise sound, according to said first anti-phase noise signal.

39. The system according to claim 38, further including an error-acoustoelectric transducer coupled with said digital active noise reduction controller,
wherein said error-acoustoelectric transducer produces an error signal by detecting said anti-phase noise sound, and
wherein said digital active noise reduction controller produces said first anti-phase noise signal, according to said error signal and said reference noise signal.

40. The system according to claim 39, further including:
an analog active noise reduction controller coupled with said error-acoustoelectric transducer; and
a summing element coupled with said analog active noise reduction controller, said digital active noise reduction controller and with said electroacoustic transducer,
wherein said analog active noise reduction controller produces a second anti-phase noise signal according to said error signal,
wherein said summing element produces a third anti-phase noise signal, by adding said first anti-phase noise signal and said second anti-phase noise signal, and
wherein said electroacoustic transducer produces said anti-phase noise sound according to said third anti-phase noise signal.

41. The system according to claim 39, wherein said electroacoustic transducer and said error-acoustoelectric transducer are acoustically separated from said reference-acoustoelectric transducer.

42. The system according to claim 38, wherein said electroacoustic transducer is acoustically separated from said reference-acoustoelectric transducer.

43. The system according to claim 38, further including:
an error-acoustoelectric transducer;
an analog active noise reduction controller coupled with said error-acoustoelectric transducer; and
a summing element coupled with said analog active noise reduction controller, said digital active noise reduction controller and with said electroacoustic transducer,
wherein said analog active noise reduction controller produces a second anti-phase noise signal according to said error signal,
wherein said summing element produces a third anti-phase noise signal, by adding said first anti-phase noise signal and said second anti-phase noise signal, and
wherein said electroacoustic transducer produces said anti-phase noise sound according to said third anti-phase noise signal.

44. The system according to claim 43, wherein said electroacoustic transducer, said reference-acoustoelectric transducer and said error-acoustoelectric transducer are coupled with a head-mounted device.

45. The system according to claim 43, wherein said error-acoustoelectric transducer includes a plurality of microphones.

46. The system according to claim 38, wherein said electroacoustic transducer includes a plurality of speakers.

47. System for producing sound, the sound including a desired sound and an anti-phase noise sound, the anti-phase noise sound being in anti-phase relative to a noise, the system comprising:
an electroacoustic transducer;
a reference-acoustoelectric transducer for producing a reference noise signal by detecting said noise in a noisy environment;
an error-acoustoelectric transducer;
a feedforward element coupled with said reference-acoustoelectric transducer;
a feedback element coupled with said feedforward element;
a first summing element coupled with said feedforward element, said feedback element and with said electroacoustic transducer;
a second summing element coupled with said feedback element, said feedforward element and with said error-acoustoelectric transducer;
a third summing element coupled with said feedback element and with said second summing element;
a first estimated plant response element coupled with said second summing element; and
a second estimated plant response element coupled with said third summing element and with said electroacoustic transducer,
wherein said first summing element produces a summation signal, by adding a feedback signal received from said feedback element, a feedforward signal received from said feedforward element, and a sound signal respective of said desired sound,
wherein said electroacoustic transducer produces said sound according to said summation signal,
wherein said first estimated plant response element produces a first estimated desired sound signal, respective of said desired sound as produced by said electroacoustic transducer,
wherein said error-acoustoelectric transducer produces an error signal by detecting said sound,
wherein said second summing element produces a first difference signal, by subtracting said first estimated desired sound signal from said error signal,
wherein said second estimated plant response element produces an estimated difference signal, according to said summation signal,
wherein said third summing element produces a second difference signal, by subtracting said estimated difference signal from said first difference signal,
wherein said feedback element produces said feedback signal according to said first difference signal and said second difference signal, and wherein said feedforward element produces said feedforward signal, according to said reference noise signal and said first difference signal.

48. The system according to claim 47, wherein said feedforward element comprises:
a feedforward estimated plant response element;
a feedforward adaptive filter; and
a feedforward least mean square element, coupled with said feedforward estimated plant response element and with said feedforward adaptive filter,
wherein said feedforward estimated plant response element and said feedforward adaptive filter receive said reference noise signal,
wherein said feedforward least mean square element receives said first difference signal, and
wherein said feedforward adaptive filter produces said feedforward signal, according to said reference noise signal, and according to a signal received from said feedforward least mean square element.

49. The system according to claim 47, wherein said feedback element includes:
a feedback estimated plant response element;
a feedback adaptive filter; and
a feedback least mean square element, coupled with said feedback estimated plant response element and with said feedback adaptive filter,
wherein said feedback least mean square element receives said first difference signal,
wherein said feedback estimated plant response element and said feedback adaptive filter receive said second difference signal, and
wherein said feedback adaptive filter produces said feedback signal, according to said second difference signal, and according to a signal received from said feedback least mean square element.

50. The system according to claim 47, wherein said electroacoustic transducer and said error-acoustoelectric transducer, are acoustically separated from said reference-acoustoelectric transducer, by a sound absorbing material.

51. The system according to claim 47, wherein said electroacoustic transducer and said error-acoustoelectric transducer are coupled with a head-mounted device.

52. The system according to claim 47, wherein said electroacoustic transducer includes a plurality of speakers.

53. The system according to claim 47, wherein said error-acoustoelectric transducer includes a plurality of microphones.

54. The system according to claim 47, wherein each of said first estimated plant response element and said first summing element receives a desired sound signal respective of said desired sound, in at least one channel.

55. Method for producing a noise-free sound signal, the method comprising the procedures of:
producing a noise bearing sound signal by detecting acoustic sound and noise;
producing a reference noise signal by detecting noise;
determining a correction signal according to said reference noise signal; and
producing said noise-free sound signal, according to said noise bearing sound signal and said correction signal.

56. The method according to claim 55, wherein said noise-free sound signal is a noise-free voice signal, and wherein said noise bearing sound signal is a noisy voice signal.

57. The method according to claim 55, wherein said procedure of determining comprises a sub-procedure of determining a reduced-intensity noise signal according to said reference noise signal, and subtracting said determined reduced-intensity noise signal from said noisy voice signal.

58. The method according to claim 57, wherein said sub-procedure of determining a reduced-intensity noise signal is performed according to at least one physical characteristic selected from the list consisting of:
hearing characteristics of another user;
voice characteristics of said user;
sound absorbing characteristics of a headset worn by said user;
distance between a reference-acoustoelectric transducer for detecting said noise, and a voice-acoustoelectric transducer for detecting said voice;
acoustic properties of the environment which surrounds said reference-acoustoelectric transducer and said voice-acoustoelectric transducer; and
acoustic properties of said reference-acoustoelectric transducer and said voice-acoustoelectric transducer.

59. The method according to claim 55, further comprising a procedure of converting said reference noise signal from analog format to digital format, after said procedure of producing a reference noise signal.

60. The method according to claim 55, further comprising a procedure of converting said noise bearing sound signal from analog format to digital format, after said procedure of producing a noise bearing sound signal.

61. The method according to claim 55, further comprising the procedures of:
determining a noise-canceling signal, according to said reference noise signal; and
producing a noise-canceling sound, according to said determined noise-canceling signal.

62. The method according to claim 61, further comprising a procedure of producing an error signal by detecting sound in the vicinity of the location of sounding said noise-canceling sound, before said procedure of determining said noise-canceling signal.

63. The method according to claim 61, wherein said procedure of producing said reference noise signal comprises a sub-procedure of determining a reduced-intensity reference noise signal, and said procedure of determining a noise-canceling signal is performed according to said reduced-intensity reference noise signal.

64. The method according to claim 55, further comprising further comprising the procedures of:
receiving an audio signal;
determining an audio-and-noise-canceling signal, according to said audio signal and said reference noise signal; and
producing an audio-and-noise-canceling sound, according to said audio-and-noise-canceling signal.

65. The method according to claim 64, wherein said procedure of producing said reference noise signal includes a sub-procedure of determining a reduced-intensity noise signal, and wherein said procedure of determining said audio-and-noise-canceling signal is performed according to said reduced-intensity noise signal.

66. The method according to claim 64, further comprising a preliminary procedure of producing an error signal, by detecting sound in the vicinity of the location of sounding said audio-and-noise-canceling sound, wherein said procedure of determining said audio-and-noise-canceling signal is performed according to said error signal.

67. Method for producing a noise-canceling sound, the method comprising the procedures of:
producing a reference noise signal by detecting noise;
determining a noise-canceling signal according to said reference noise signal; and
producing said noise-canceling sound according to said determined noise-canceling signal.

68. The method according to claim 67, wherein said procedure of producing said reference noise signal includes a sub-procedure of determining a reduced-intensity noise signal, and wherein said procedure of determining is performed according to said reduced-intensity noise signal.

69. The method according to claim 67, further comprising a preliminary procedure of producing an error signal, by detecting sound in the vicinity of the location of sounding said noise-canceling sound, wherein said procedure of determining said noise-canceling signal is performed according to said error signal.

70. Method for producing an audio-and-noise-canceling sound, the method comprising the procedures of:
producing a reference noise signal by detecting noise;
receiving an audio signal;
determining an audio-and-noise-canceling signal according to said reference noise signal and said audio signal; and
producing said audio-and-noise-canceling sound according to said determined audio-and-noise-canceling signal.

71. The method according to claim 70, wherein said procedure of producing said reference noise signal includes a sub-procedure of determining a reduced-intensity noise signal, and wherein said procedure of determining is performed according to said reduced-intensity noise signal.

72. The method according to claim 70, further comprising a preliminary procedure of producing an error signal, by detecting sound in the vicinity of the location of sounding said audio-and-noise-canceling sound, wherein said procedure of determining said audio-and-noise-canceling signal is performed according to said error signal.
